(19) United States
(12) Patent Application Publication
(10) Pub. No.: US 2021/0267623 A1
(43) Pub. Date: Sep. 2, 2021

(54) ULTRASONIC TRANSDUCER ASSEMBLY FOR A SURGICAL INSTRUMENT AND METHOD OF ASSEMBLING THE SAME

(71) Applicant: Covidien LP, Mansfield, MA (US)

(72) Inventors: Matthew S. Cowley, Frederick, CO (US); Titus Horton, Lyons, CO (US); David J. Van Tol, Boulder, CO (US); James Ryan Fagan, Erie, CO (US); Weng-Kai Lee, Longmont, CO (US); Christopher T. Tschudy, Arvada, CO (US)

(21) Appl. No.: 17/187,250

(22) Filed: Feb. 26, 2021

Related U.S. Application Data
(60) Provisional application No. 62/983,411, filed on Feb. 28, 2020.

(51) Int. Cl. A61B 17/32

(52) U.S. Cl. CPC A61B 17/320092; A61B 2017/00402

(57) ABSTRACT

A ultrasonic transducer assembly of an ultrasonic surgical instrument includes a horn, a piezoelectric stack positioned proximally of the horn, a proximal end mass positioned proximally of the piezoelectric stack, and a rod extending through the piezoelectric stack. The rod includes a proximal end portion directly secured to the proximal end mass and a distal end portion directly secured to the horn to thereby maintain the piezoelectric stack in compression against the horn.

### CROSS-REFERENCE TO RELATED APPLICATIONS

This application claims the benefit of and priority to U.S. Provisional Patent Application Ser. No. 62/983,411 filed Feb. 28, 2020, the entire disclosure of which is incorporated by reference herein.

### BACKGROUND

### Technical Field

The present disclosure relates to ultrasonic surgical instruments and, more particularly, to an ultrasonic transducer assembly for an ultrasonic surgical instrument and method of assembling the same.

### Background of Related Art

Ultrasonic surgical instruments utilize ultrasonic energy, i.e., ultrasonic vibrations, to treat tissue. More specifically, ultrasonic surgical instruments utilize mechanical vibration energy transmitted at ultrasonic frequencies to coagulate, cauterize, fuse, seal, cut, desiccate, fulgurate, and/or otherwise treat tissue.

Ultrasonic surgical instruments typically employ a transducer coupled to a handle of the ultrasonic surgical instrument and configured to produce ultrasonic energy for transmission along a waveguide to an end effector of the ultrasonic surgical instrument that is designed to treat tissue with the ultrasonic energy. The transducer may be driven by an ultrasonic generator that is on-board, e.g., on or within the handle of the ultrasonic surgical instrument, or remotely disposed, e.g., as a set-top box connected to the ultrasonic surgical instrument via an electrical cable. The end effector of the ultrasonic surgical instrument may include a blade that receives the ultrasonic energy from the waveguide for application to tissue and a jaw member configured to clamp tissue between the blade and the jaw member to facilitate treatment thereof.

### SUMMARY

As used herein, the term “distal” refers to the portion that is described which is further from a user, while the term “proximal” refers to the portion that is being described which is closer to a user. Further, any or all of the aspects described herein, to the extent consistent, may be used in conjunction with any or all of the other aspects described herein.

Provided in accordance with aspects of the present disclosure is an ultrasonic transducer assembly of an ultrasonic surgical instrument including a horn, a piezoelectric stack positioned proximally of the horn, a proximal end mass positioned proximally of the piezoelectric stack, and a rod extending through the piezoelectric stack. The rod includes a proximal end portion directly secured to the proximal end mass and a distal end portion directly secured to the horn to thereby maintain the piezoelectric stack in compression against the horn.

In an aspect of the present disclosure, the proximal end portion of the rod is fused, e.g., welded, brazed, diffusion bonded, adhered, etc., to the proximal end mass.

In another aspect of the present disclosure, the distal end portion of the rod is received within a proximally-facing cavity defined within the horn.

In still another aspect of the present disclosure, an electrode assembly including at least one electrode contacting a surface of at least one piezoelectric element of the piezoelectric stack and at least one electrode contacting an opposed surface of the at least one piezoelectric element of the piezoelectric stack is provided.

In yet another aspect of the present disclosure, a distal end mass disposed between the piezoelectric stack and the horn. In such aspects, the piezoelectric stack is maintained in compression against the horn with the distal end mass disposed therebetween.

In still yet another aspect of the present disclosure, a casing at least partially encloses the horn, the piezoelectric stack, the proximal end mass, and the rod therein.

In another aspect of the present disclosure, the piezoelectric stack in maintained in compression against the horn at a pre-determined compression force or voltage or at a compression force or voltage within a pre-determined compression force or voltage range, respectively. To achieve this pre-determined compression force/voltage or compression force/voltage within a pre-determined compression force/voltage range, the piezoelectric stack may be compressed to achieve a pre-determined voltage by the piezoelectric stack or a voltage within a pre-determined voltage range, and thereafter secured in position. Alternatively, the piezoelectric stack may be compressed to a compression force or compression force within a compression force range (similar to or different from the pre-determined compression force/range) and thereafter secured in position.

A method of assembling an ultrasonic transducer assembly in accordance with aspects of the present disclosure includes arranging a piezoelectric stack, a horn, a proximal end mass, and a rod to form an assembly whereby a distal end portion of the rod is secured to the horn, the piezoelectric stack is disposed about the rod proximally of the horn, and the end mass is disposed about the rod proximally of the piezoelectric stack. The method further includes applying a longitudinal compressive force distally from the proximal end mass and/or proximally from the horn to compress the piezoelectric stack between the proximal end mass and the horn. The method further includes securing a proximal end portion of the rod to the proximal end mass to maintain the piezoelectric stack under compression.

In an aspect of the present disclosure, securing the proximal end portion of the rod to the proximal end mass includes fusing, e.g., welding, the proximal end portion of the rod to the proximal end mass in at least one location.

In another aspect of the present disclosure, the piezoelectric stack is maintained under a pre-determined compression force or voltage and/or a compression force or voltage within a pre-determined compression force or voltage range, respectively. To achieve this pre-determined compression force/voltage or compression force/voltage within a pre-determined compression force/voltage range, the piezoelectric stack may be compressed to achieve a pre-determined voltage by the piezoelectric stack or a voltage within a pre-determined voltage range, and thereafter secured in position. Alternatively, the piezoelectric stack may be compressed to a compression force or compression force within a compression force range (similar to or different from the pre-determined compression force/range) and thereafter secured in position.

In yet another aspect of the present disclosure, the method includes installing the assembly in a fixture prior to applying the longitudinal compressive force. In such aspects, the fixture may retain the assembly at a distal location on the horn and a proximal location on the proximal end mass and/or may apply the longitudinal compressive force to at least one of the proximal location or the distal location.

In another aspect of the present disclosure, the arranging further includes positioning at least one electrode about the rod and in contact with a surface of at least one piezoelectric element of the piezoelectric stack and positioning at least one electrode about the rod and in contact with an opposed surface of the at least one piezoelectric element of the piezoelectric stack.

In still another aspect of the present disclosure, the arranging further includes positioning a distal end mass about the rod and between the piezoelectric stack and the horn.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a side, perspective view of an ultrasonic surgical instrument provided in accordance with the present disclosure;

FIG. 2 is an enlarged, side, longitudinal, cross-sectional view of a proximal portion of the ultrasonic surgical instrument of FIG. 1;

FIG. 3 is a longitudinal, cross-sectional view of the ultrasonic transducer assembly of ultrasonic surgical instrument of FIG. 1;

FIG. 4 is an exploded, longitudinal, cross-sectional view of the ultrasonic transducer assembly of ultrasonic surgical instrument of FIG. 1; and

FIG. 5 is a longitudinal, cross-sectional view illustrating assembly of the ultrasonic transducer assembly of ultrasonic surgical instrument of FIG. 1.

### DETAILED DESCRIPTION

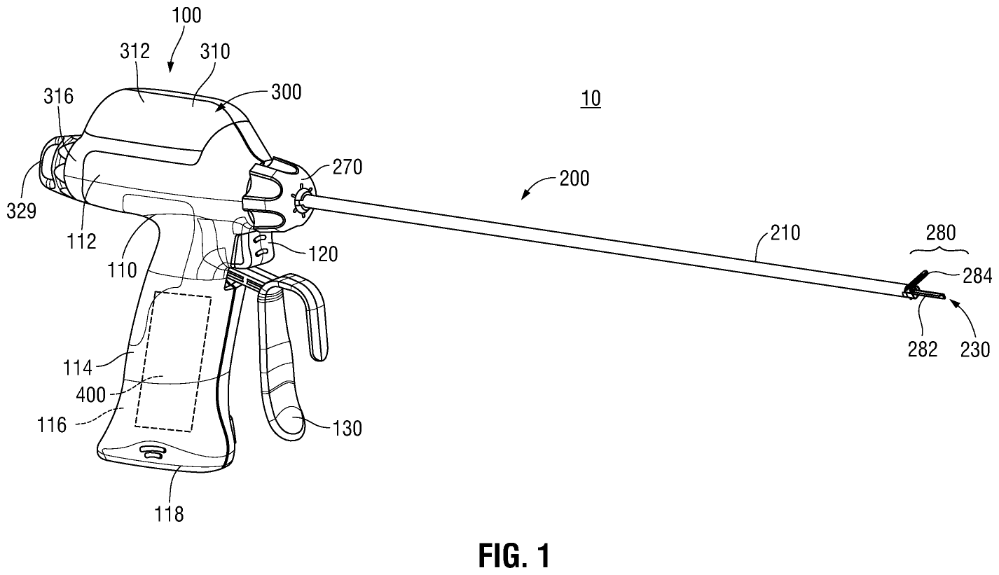

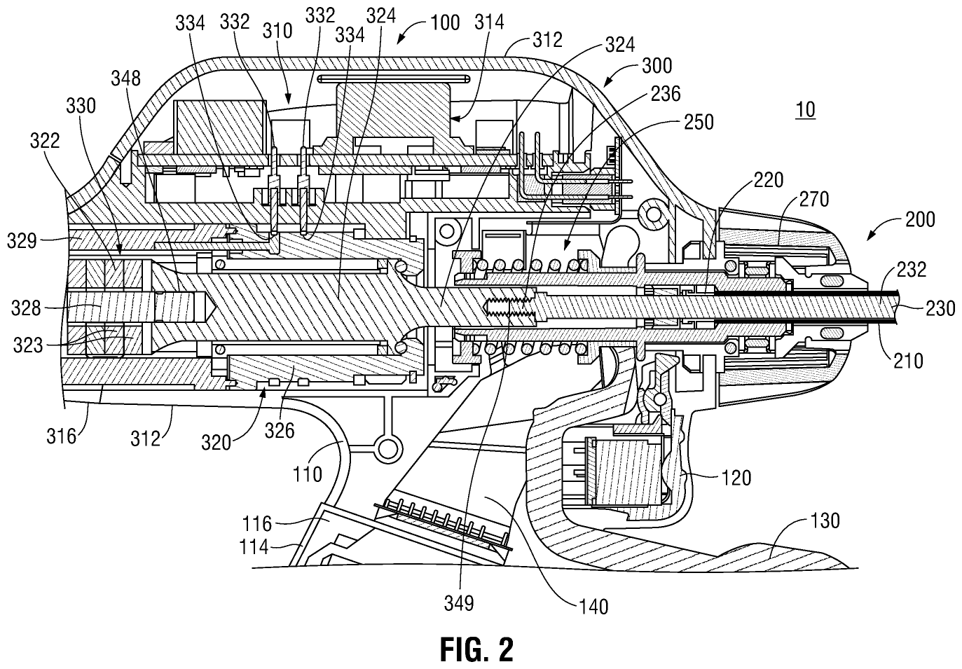

Referring to FIGS. 1 and 2, an ultrasonic surgical instrument provided in accordance with aspects of the present disclosure is shown generally identified by reference numeral 10. Ultrasonic surgical instrument 10 includes a handle assembly 100 and an elongated assembly 200 extending distally from handle assembly 100. Handle assembly 100 includes a housing 110 defining a body portion 112 and a fixed handle portion 114. Handle assembly 100 further includes an activation button 120 and a clamp trigger 130.

Body portion 112 of housing 110 is configured to support an ultrasonic transducer and generator assembly (“TAG”) 300 including a generator assembly 310 and an ultrasonic transducer assembly 320. TAG 300 may be permanently engaged with body portion 112 of housing 110 or removable therefrom. Generator assembly 310 includes a housing 312 configured to house the internal electronics 314 of generator assembly 310, and a cradle 316 configured to rotatably support ultrasonic transducer assembly 320. Alternatively, generator assembly 310 may be remotely disposed and coupled to ultrasonic surgical instrument 10 by way of a surgical cable.

Ultrasonic transducer assembly 320 generally includes a piezoelectric stack 322, a horn 324, a casing 326, and an electrode assembly 330. Ultrasonic transducer 320 also includes a rotation knob 329. Casing 326 and rotation knob 329 are engaged with one another and cooperate to form an enclosure to encapsulate the internal components of ultrasonic transducer assembly 320 with a portion of horn 324 extending distally from casing 326. Rotation knob 329 is accessible from the exterior of handle assembly 100 and is configured for manual rotation to rotate ultrasonic transducer assembly 320 relative to generator assembly 310 and housing 110.

Continuing with reference to FIGS. 1 and 2, a set of connectors 332 and corresponding rotational contacts 334 associated with generator assembly 310 and ultrasonic transducer assembly 320, respectively, enable drive signals to be communicated from generator assembly 310 to piezoelectric stack 322 of ultrasonic transducer assembly 320 to drive ultrasonic transducer assembly 320 regardless of the rotational orientation of ultrasonic transducer assembly 320. More specifically, connectors 332 and rotational contacts 334 enable a drive signal voltage to be applied from generator assembly 310 across piezoelectric stack 322 via the positive and negative electrodes of electrode assembly 330. The piezoelectric stack 322, in turn, converts the applied voltage into mechanical energy, in the form of ultrasonic vibrations, that is transmitted to ultrasonic horn 324 (via the compression engagement therebetween, as detailed below). Horn 324, in turn, is configured to transmit the ultrasonic energy produced by piezoelectric stack 322 to waveguide 230 of elongated assembly 200 for transmission therealong to blade 282 of end effector 280 of elongated assembly 200.

Referring still to FIGS. 1 and 2, fixed handle portion 114 of housing 110 defines a compartment 116 configured to receive a battery assembly 400 and a door 118 configured to enclose compartment 116. An electrical connection assembly 140 is disposed within housing 110 of handle assembly 100 and serves to electrically couple activation button 120, generator assembly 310 of TAG 300, and battery assembly 400 with one another when TAG 300 is supported on or in body portion 112 of housing 110 and battery assembly 400 is disposed within compartment 116 of fixed handle portion 114 of housing 110, thus enabling activation of ultrasonic surgical instrument 10 in response to depression of activation button 120. More specifically, when activation button 120 is activated in an appropriate manner, an underlying two-mode switch assembly of activation button 120 is activated to supply power from the battery assembly 400 to TAG 300 in either a “LOW” power mode or a “HIGH” power mode, depending upon the manner of activation of activation button 120. Activation button 120 may move in the same direction from an off position, to the “LOW” power mode, to the “HIGH” power mode or may move therebetween in any other suitable manner. Alternatively, separate activation buttons and/or different modes of operation are also contemplated.

In embodiments where generator assembly 310 is remote from ultrasonic surgical instrument 10, battery assembly 400 and the configuration of fixed handle portion 114 for receiving battery assembly 400 need not be provided, as generator assembly 310 may be powered by a standard wall outlet or other power source.

Elongated assembly 200 of ultrasonic surgical instrument 10 includes an outer drive sleeve 210, an inner support sleeve 220 disposed within outer drive sleeve 210, a waveguide 230 extending through inner support sleeve 220, a drive assembly 250, a rotation knob 270, and an end effector 280 including a blade 282 and a jaw 284. A proximal portion of outer drive sleeve 210 is operably coupled to clamp trigger 130 of handle assembly 100 via drive assembly 250, while a distal portion of outer drive sleeve 210 is operably coupled to jaw 284. As such, clamp trigger 130 is selectively actuatable to thereby move outer drive sleeve 210 about inner support sleeve 220 to pivot jaw 284 relative to blade 282 of end effector 280 from a spaced-apart position to an approximated position for clamping tissue between jaw 284 and blade 282. Drive assembly 250 provides a force-limiting feature whereby the clamping pressure applied to tissue is limited to a particular clamping pressure or particular clamping pressure range. Rotation knob 270 is rotatable in either direction to rotate elongated assembly 200 in either direction relative to handle assembly 100.

Waveguide 230, as noted above, extends through inner support sleeve 220. Waveguide 230 defines a body 232 and a blade 282 extending from the distal end of body 232. Blade 282 serves as the blade of end effector 280. Waveguide 230 further includes a proximal threaded male connector 236 configured for threaded engagement within threaded female receiver 349 of horn 324 such that ultrasonic motion produced by ultrasonic transducer assembly 320 is transmitted along waveguide 230 to blade 282 for treating tissue clamped between blade 282 and jaw 284 or positioned near blade 282. Other suitable engagements between waveguide 230 and horn 324 are also contemplated.

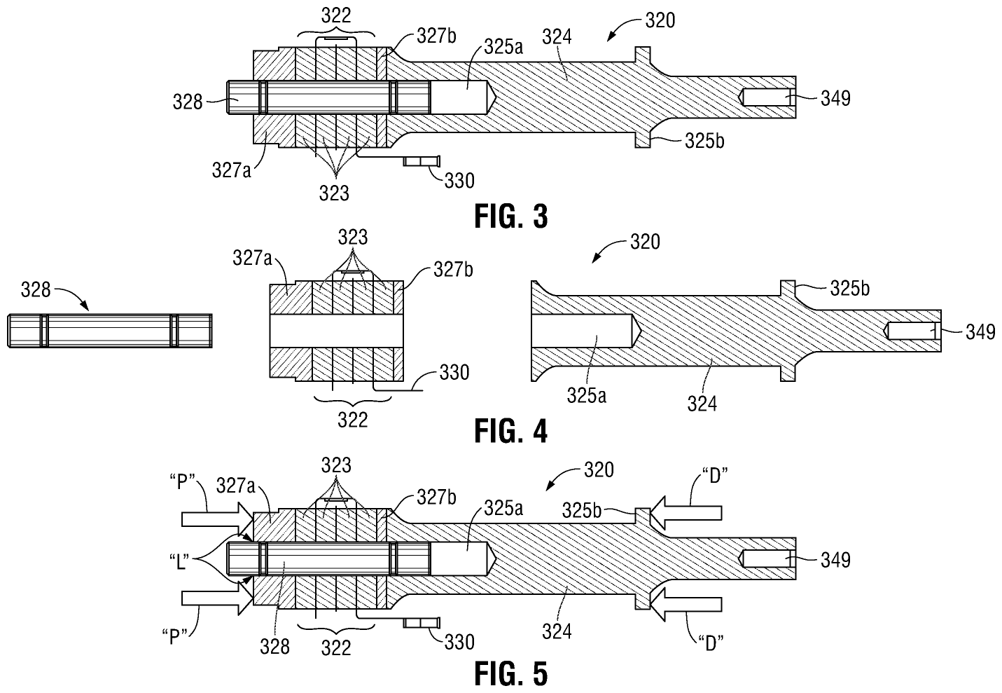

Turning now to FIG. 3, ultrasonic transducer assembly 320, more specifically, includes piezoelectric stack 322, horn 324, casing 326 (FIG. 2), proximal and distal end masses 327a, 327b, and a rod 328 securing piezoelectric stack 322 between proximal and distal end masses 327a, 327b, respectively, and to horn 324 under compression. In embodiments, distal end mass 327b is omitted and piezoelectric stack 322 is compressed directly against horn 324. In either configuration, the pre-compression of piezoelectric stack 322 against horn 324 (directly or indirectly), enables efficient and effective transmission of ultrasonic energy from piezoelectric stack 322 to horn 324 for transmission along waveguide 230 to blade 282 for treating tissue clamped between blade 282 and jaw 284 or positioned near blade 282 (see FIGS. 1 and 2). Ultrasonic transducer assembly 320 further includes electrode assembly 330 having at least one electrode disposed in contact with a surface of at least one piezoelectric element 323 of piezoelectric stack 322 and at least one electrode disposed in contact with an opposed surface of the at least one piezoelectric element 323 of piezoelectric stack 322 to, as noted above, enable a drive signal voltage to be applied across piezoelectric stack 322.

Rod 328, as noted above, secures piezoelectric stack 322 between proximal and distal end masses 327a, 327b, respectively, and to horn 324 under compression. This is accomplished via securement of rod 328 at a distal end portion thereof to horn 324 and at a proximal end portion thereof to proximal end mass 327a, without the need for additional securement structures such as, for example, a proximal nut. In embodiments, rod 328 and horn 324 are integrally formed as a single, monolithic component prior to assembly of ultrasonic transducer assembly 320. In such embodiments, securement of rod 328 at a distal end portion thereof to horn 324 during assembly is not required as rod 328 is already formed with horn 324.

Referring to FIGS. 4 and 5, to assemble ultrasonic transducer assembly 320, the electrodes of electrode assembly 330 are interdisposed between piezoelectric elements 323 of piezoelectric stack 322 and proximal and distal end masses 327a, 327b are arranged at the proximal end distal ends, respectively, of piezoelectric stack 322. A distal end portion of rod 328 is secured within a proximally-facing cavity 325a defined within horn 324, e.g., via threaded engagement, welding, press-fitting, combinations thereof, or in any other suitable manner. Alternatively, as noted above, rod 328 and horn 324 may be integrally formed, obviating the need for this securement. Further still, in other embodiments, rod 328 may be integrally formed with distal end mass 327b. The electrodes of electrode assembly 330, piezoelectric elements 323, and proximal and distal end masses 327a, 327b are disposed about rod 328 and positioned such that distal end mass 327b abuts horn 324. Some or all of the above steps may be performed in the order detailed above, in any other suitable order, simultaneously, and/or in overlapping temporal relation relative to one another.

Regardless of the particular order of steps, the above results in a configuration wherein the distal end portion of rod 328 is secured within proximally-facing cavity 325a of horn 324 and distal end mass 327b, piezoelectric stack 322 (including the electrodes of electrode assembly 330 interdisposed between piezoelectric elements 323 of piezoelectric stack 322), and proximal end mass 327a are disposed about rod 328 in a distal-to-proximal direction extending from horn 324. Once this position has been achieved, the assembly may be loaded into a fixture whereby the fixture contacts and retains the assembly at a distal location such as location “D,” e.g., near annular flange 325b of horn 324 or at any other suitable location or locations sufficient to retain horn 324, and at a proximal location such as location “P,” e.g., at proximal end mass 327a.

With the fixture retaining the assembly at locations “P” and “D,” the fixture may then be manipulated, actuated, or otherwise adjusted to apply a longitudinal compressive force to the assembly, e.g., at the distal location “D” and/or the proximal location “P.” More specifically, the longitudinal compressive force is applied until a suitable pre-compression of piezoelectric stack 322 between proximal end mass 327a and horn 324 is achieved. As an alternative, a longitudinal force may be applied distally at the proximal location “P” (or a holding force may be applied to maintain position), while a proximal end portion of rod 328 is pulled proximally to apply a longitudinal force in the proximal direction, until a suitable pre-compression of piezoelectric stack 322 between proximal end mass 327a and horn 324 is achieved. In such a configuration, there is reduced or no relaxation of the pre-compression applied during assembly since rod 328 is already stretched (as a result of the proximal pulling of the proximal end portion thereof) during assembly. Regardless of the manner of pre-compression, since the piezoelectric stack 322 produces a voltage as it is compressed, the longitudinal compressive force may be applied until a pre-determined voltage by the piezoelectric stack 322 is measured or until a voltage within a pre-determined voltage range is measured. This voltage or voltage range may correspond to a pre-determined pre-compression force or pre-compression force range. Alternatively, a force gauge may be utilized to determine the pre-compression force.

Continuing with reference to FIG. 5, once the desired pre-compression force (or a pre-compression force within a desired range) is reached, the fixture is locked or otherwise maintained to retain the assembly in position, thereby maintaining the piezoelectric stack 322 between proximal end mass 327a and horn 324 under compression. Thereafter, the proximal end portion of rod 328 is secured to proximal end mass 327a, for example, by fusing, e.g., welding, in one or more locations “L” at the interface between rod 328 and proximal end mass 327a, e.g., in one or more annular positions about the circumference of rod 328, although additional or alternative fuse locations and/or manners of securement are also contemplated. It is noted that the pre-determined pre-compression force or compression force within a pre-determined compression force range applied during assembly does not necessarily equal the maintained compression force, e.g., due to expansion or stretching of component(s) upon release, temperature, etc.; thus, the pre-determined pre-compression force or pre-compression force within a pre-determined pre-compression range may be selected to achieve a resultant maintained compression force.

With the proximal end portion of rod 328 fused, e.g., welded, or otherwise secured to proximal end mass 327a, the assembly may be removed from the fixture. Finally, casing 326 and rotation knob 329 may be positioned about the assembly and secured thereabout to complete the assembly of ultrasonic transducer assembly 320.

While several embodiments of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular embodiments. Those skilled in the art will envision other modifications within the scope and spirit of the claims appended hereto.

What is claimed is:

1. An ultrasonic transducer assembly of an ultrasonic surgical instrument, comprising:
a horn;
a piezoelectric stack positioned proximally of the horn;
a proximal end mass positioned proximally of the piezoelectric stack; and
a rod extending through the piezoelectric stack, the rod including a proximal end portion directly secured to the proximal end mass and a distal end portion directly secured to the horn to thereby maintain the piezoelectric stack in compression against the horn.

2. The ultrasonic transducer assembly according to claim 1, wherein the proximal end portion of the rod is fused to the proximal end mass.

3. The ultrasonic transducer assembly according to claim 1, wherein the distal end portion of the rod is received within a proximally-facing cavity defined within the horn.

4. The ultrasonic transducer assembly according to claim 1, further comprising an electrode assembly including at least one electrode disposed in contact with a surface of at least one piezoelectric element of the piezoelectric stack and at least one electrode disposed in contact with an opposed surface of the at least one piezoelectric element of the piezoelectric stack.

5. The ultrasonic transducer assembly according to claim 1, further comprising a distal end mass disposed between the piezoelectric stack and the horn, wherein the piezoelectric stack is maintained in compression against the horn with the distal end mass disposed therebetween.

6. The ultrasonic transducer assembly according to claim 1, further comprising a casing at least partially enclosing the horn, the piezoelectric stack, the proximal end mass, and the rod therein.

7. The ultrasonic transducer assembly according to claim 1, wherein the piezoelectric stack in maintained in compression against the horn at a pre-determined compression force or voltage.

8. The ultrasonic transducer assembly according to claim 1, wherein the piezoelectric stack in maintained in compression against the horn at a compression force or voltage within a pre-determined compression force or voltage range, respectively.

9. A method of assembling an ultrasonic transducer assembly, comprising:
arranging a piezoelectric stack, a horn, a proximal end mass, and a rod to form an assembly whereby a distal end portion of the rod is secured to the horn, the piezoelectric stack is disposed about the rod proximally of the horn, and the end mass is disposed about the rod proximally of the piezoelectric stack;
applying a longitudinal compressive force at least one of distally from the proximal end mass or proximally from the horn to compress the piezoelectric stack between the proximal end mass and the horn; and
securing a proximal end portion of the rod to the proximal end mass to maintain a compression force on the piezoelectric stack between the proximal end mass and the horn.

10. The method according to claim 9, wherein securing the proximal end portion of the rod to the proximal end mass includes fusing the proximal end portion of the rod to the proximal end mass in at least one location.

11. The method according to claim 9, wherein the compression force is a pre-determined compression force or voltage.

12. The method according to claim 9, wherein the compression force is within a pre-determined compression force or voltage range.

13. The method according to claim 9, further comprising installing the assembly in a fixture prior to applying the longitudinal compressive force.

14. The method according to claim 13, wherein the fixture retains the assembly at a distal location on the horn and a proximal location on the proximal end mass.

15. The method according to claim 14, wherein the fixture applies the longitudinal compressive force to at least one of the proximal location or the distal location.

16. The method according to claim 9, wherein arranging further includes positioning at least one positive electrode about the rod and between a first pair of piezoelectric elements of the piezoelectric stack and positioning at least one negative electrode about the rod and between a second pair of piezoelectric elements of the piezoelectric stack.

17. The method according to claim 9, wherein arranging further includes positioning a distal end mass about the rod and between the piezoelectric stack and the horn.
